(19) United States
(12) Patent Application Publication
(10) Pub. No.: US 2006/0159637 A1
(43) Pub. Date: Jul. 20, 2006

(54) Surface-modified, structurally modified titanium dioxides

(76) Inventors: Jurgen Meyer, Stockstadt (DE); Steffen Hasenzahl, Morris Plains, NJ (US); Heike Riedemann, Mombris (DE); Ann Gray, Hanau (DE)

Correspondence Address: SMITH, GAMBRELL & RUSSELL, LLP, 1230 PEACHTREE STREET, N.E., SUITE 3100, PROMENADE II, ATLANTA, GA, 30309-3592, US

(21) Appl. No.: 11/316,591

(22) Filed: Dec. 22, 2005

(30) Foreign Application Priority Data
Dec. 23, 2004 (EP) 04 030 588.0

(51) Int. Cl. A61K 8/29; A61K 8/89

(52) U.S. Cl. 424/59; 424/70.12

(57) ABSTRACT

Pyrogenically prepared, surface-modified, structurally modified titanium dioxides and pyrogenically prepared, surface-modified, structurally modified titanium dioxide mixed oxides are prepared by structurally modifying and then surface modifying the titanium dioxide or titanium dioxide mixed oxide. The titanium dioxides can be used to produce sunscreen formulations.

### INTRODUCTION AND BACKGROUND

The invention provides pyrogenically prepared, surface-modified, structurally modified titanium dioxides or pyrogenically prepared, surface modified, structurally modified titanium dioxide/iron oxide mixed oxides, a process for the preparation thereof and the use thereof in sunscreen formulations.

The use of pyrogenically prepared titanium dioxide and a pyrogenically prepared titanium dioxide/iron oxide mixed oxide in sunscreen formulations has been disclosed. These mixed oxides are not structurally modified (EP 0639533).

The known pyrogenically prepared titanium dioxides have the disadvantage that they are not sufficiently transparent in the sunscreen formulations prepared therewith when these sunscreen formulations are applied to the skin.

When preparing the sunscreen formulation, a costly and disadvantageous dispersion process is required. During the dispersion procedure, an intense thickening effect in cosmetic oils or water hinders the production of dispersions or sunscreen agents with a high titanium dioxide content.

Disadvantageously, the sunscreen formulations prepared with the known titanium dioxides feel rough on the skin.

The object of the invention is to produce pyrogenically prepared titanium dioxides which have better transparency in sunscreen agents and also feel nicer on the skin.

### SUMMARY OF THE INVENTION

The invention provides pyrogenically prepared, surface modified, structurally modified titanium dioxides or pyrogenically prepared, surface-modified, structurally modified titanium dioxide mixed oxides.

The invention also provides a process for preparing the pyrogenically prepared, surface-modified, structurally modified titanium dioxides or the pyrogenically prepared, surface-modified, structurally modified titanium dioxide mixed oxides, which is characterised in that pyrogenically prepared titanium dioxide or pyrogenically prepared titanium dioxide mixed oxide is surface-modified and then structurally modified using a ball mill and optionally post-milled.

### DETAILED DESCRIPTION OF INVENTION

The pyrogenically prepared titanium dioxides listed in Table 1, for example, may be used as educts or starting materials.

TABLE 1

| | Aeroxide ® | Aeroxide ® | Aeroxide ® |
|---|---|---|---|
| | TiO₂ P25 | T1O₂ P25 S | TiO₂ PF2 |
| Appearance | white | white | white |
| | powder | powder | powder |
| Spec. surface area | 50 ± 15 | 50 ± 15 | 57.5 ± 12.5 |
| (BET)¹⁾ m²/g | | | |
| Av. size of primary | 21 | — | — |
| particles nm | | | |
| Compacted bulk density | 130 | — | 80 |
| (approx. value)²⁾ g/l | | | |
| Bulk density (approx. | — | 60-150 | — |
| value) g/l | | | |
| Loss on drying³⁾ | ≦1.5 | — | ≦2.0 |
| (2 hrs. 105° C.) % | | | |
| Loss on ignition⁴⁾⁷⁾ | ≦2.0 | — | ≦3.0 |
| (2 hrs. 1000° C.) % | | | |
| pH⁵⁾ | 3.5-4.5 | — | 3.5-4.5 |
| As content ppm | — | ≦1.0 | — |
| Hg content ppm | — | ≦1.0 | — |
| Sb content ppm | — | ≦2.0 | — |
| Pb content ppm | — | ≦10 | — |
| SiO₂ content⁸⁾ wt. % | ≦0.200 | — | — |
| Al₂O₃ content⁸⁾ wt. % | ≦0.300 | — | — |
| Fe₂O₃ content⁸⁾ wt. % | ≦0.010 | — | 2.0 ± 1 |
| TiO₂ content⁸⁾ wt. % | ≧99.5 | ≧99.0; | ≧94.00 |
| | | ≦100.5 | |
| HCl content¹⁰⁾ wt. % | ≦0.300 | ≦0.3 | ≦0.800 |
| Sieve residue⁶⁾ wt. % | ≦0.050 | — | — |
| (Mocker's method, | | | |
| 45 μm) | | | |

¹⁾According to DIN 66131
²⁾According to DIN EN ISO 787-11, JIS K 5101/20 (unsieved)
³⁾According to DIN EN ISO 787-2, ASTM D280, JIS K 5101/23
⁴⁾According to DIN EN ISO 3262-20, ASTM D 1208, JIS K 5101/24
⁵⁾According to DIN EN ISO 787-9, ASTM D 1208, JIS K 5101/26
⁶⁾According to DIN EN ISO 787-18, JIS K 5101/22
⁷⁾Based on dried substance (2 hrs. at 105° C.)
⁸⁾Calcined substance (2 hrs. at 1000° C.)
⁹⁾Special moisture-proof packaging
¹⁰⁾HCl content is part of the loss on ignition

In particular, an iron oxide-containing titanium dioxide powder can be used as the starting material, this being disclosed in the document EP 0 609 533 A1.

The iron oxide-containing titanium dioxide powder may comprise a pyrogenically prepared iron oxide/titanium dioxide mixed oxide with a BET surface area of 10 to 150 m²/g which contains 0.5 to 50 wt % of iron oxide, with respect to the total amount, as a constituent of the mixed oxide.

This may be prepared by evaporating anhydrous iron(III) chloride, transferring this, together with an inert gas, for example nitrogen, into the mixing chamber of a known burner, mixing there with hydrogen, air and gaseous titanium tetrachloride in a ratio that corresponds to the composition of the iron oxide/titanium dioxide mixed oxide, burning the 4-component mixture in a reaction chamber, then separating the solid iron oxide/titanium dioxide mixed oxide from the gaseous reaction products and optionally removing adhering hydrogen chloride therefrom in moist air.

The pyrogenic preparation of titanium dioxide P25 is disclosed in Ullmann's Enzyklopädie der technischen Chemie vol. 21, 4th edition (1982) page 464.

Titanium dioxide P 25 is prepared by the flame hydrolysis of titanium tetrachloride in accordance with the equation
TiCl₄+2H₂+O₂→TiO₂+4HCl.

Titanium dioxide PF 2, which is a titanium dioxide doped with 2% iron oxide, is also prepared by this method in accordance with the equation
TiCl₄+2H₂+O₂→TiO₂+4HCl
and
2FeCl₃+3H₂+1.5O₂→Fe₂O₃+6HCl.

Crystallographically, the two products titanium dioxide PF 2 and P 25 consist of about 80% anatase and 20% rutile. They have an average primary particle size of about 20 nm.

The physico-chemical properties of titanium dioxide PF2 and P25 are summarised in Table 1.

Titanium dioxide P 25 S has the physico-chemical properties listed in Table 1.

Furthermore, a pyrogenically prepared titanium dioxide in accordance with DE 103 57 508.1 can be used as a starting material, this being present in aggregates of primary particles, and is characterised in that
it has a BET surface area of 20 to 200 m²/g and
the half-width HW, in nanometres, of the primary particle distribution has a value of
HW [nm]=a×BET^{f}, where a=670×10⁹ m³/g and −1.3≦f≦−1.0, and
the proportion of particles with a diameter of more than 45 μm is within the range 0.0001 to 0.05 wt. %.

It can be prepared by

evaporating a titanium halide, preferably titanium tetrachloride, at temperatures of less than 200° C., transferring the vapour into a mixing chamber using a carrier gas with a proportion of water vapour within the range 1 to 25 g/m³,

separately transferring hydrogen, primary air, which may be optionally enriched with oxygen and/or may be preheated, and water vapour into the mixing chamber,

wherein the proportion of water vapour is within the range 1 to 25 g/m³ of primary air,

the lambda value being in the range 1 to 9 and the gamma value being in the range 1 to 9,

then

igniting the mixture consisting of the vapour of titanium halide, hydrogen, air and water vapour in a burner and burning the flame in a reaction chamber from which ambient air is excluded, wherein

there is a vacuum of 1 to 200 mbar in the reaction chamber and the rate of emergence of the reaction mixture from the mixing chamber into the reaction chamber can be within the range 10 to 80 m/s,

additional secondary air is introduced into the reaction chamber, wherein

the ratio primary air/secondary air may be between 10 and 0.5,

then the solids are separated from gaseous substances and

the solids are then treated with water vapour.

Surface modification can be performed by spraying the oxides optionally first of all with water and then with the surface modification agent. The water used can be acidified to a pH of 7 to 1 with an acid, for example hydrochloric acid. If several surface modification agents are used, these may be applied together or separately, in sequence or as a mixture. The surface modification agent(s) can be dissolved in suitable solvents. After completion of the spray process, mixing can be continued for another 5 to 30 minutes.

The mixture is then subjected to thermal treatment at a temperature of 20 to 400° C. for a period of 0.1 to 6 hours. Thermal treatment may be performed under a protective gas such as, for example, nitrogen.

An alternative method for surface modification of the oxides can be performed by treating the oxides with the surface modification agent in vapour form and then subjecting the mixture to thermal treatment at a temperature of 50 to 800° C. for a period of 0.1 to 6 hours. Thermal treatment may be performed under a protective gas such as, for example, nitrogen. Thermal treatment may also be performed in several stages at different temperatures.

The surface modification agent(s) can be applied using one-component, two-component or ultrasonic nozzles.

Surface modification can be performed continuously or batchwise in heatable mixers and dryers with spray units. Suitable devices are, for example: plough bar mixers or disc, fluidised bed or moving bed dryers.

Structural modification of the silicas prepared in this way is then performed by mechanical means. Post-milling may be performed, if required, after structural modification. A conditioning process may be performed, if required, after structural modification and/or post-milling.

Structural modification can be performed, for example, with a ball mill or a continuously operating ball mill. Post-milling can be performed, for example, using a compressed air mill, a toothed-disc mill or a pin mill.

Conditioning may be performed batchwise, for example in a drying cabinet, or continuously, for example in a moving or fluidised bed. Conditioning may be performed under a protective gas, for example nitrogen.

The following may be used as surface modification agents:

a) organosilanes of the type (RO)₃Si(CₙH₂ₙ₊₁) and
(RO)₃Si(CₙH₂ₙ₋₁)
   R=alkyl, such as for example methyl, ethyl, n-propyl,
   i-propyl, butyl-
   n=1-20

b) organosilanes of the type R′ₓ(RO)_{y}Si(CₙH₂ₙ₊₁) and
R′x(RO)ySi(CₙH₂ₙ₋₁)
R=alkyl, such as for example methyl, ethyl, n-propyl, i-propyl, butyl
R′=alkyl, such as for example methyl, ethyl,
n-propyl, i-propyl, butyl
R′=cycloalkyl
n=1-20
x+y=3
x=1,2
y=1,2

c) halogeno-organosilanes of the type X₃Si(CnH₂ₙ₊₁) and X₃Si(CₙH₂ₙ₋₁)
X=Cl, Br
n=1-20

d) halogeno-organosilanes of the type X₂(R′)Si(CₙH₂ₙ₊₁) and X₂(R′)Si(CₙH₂ₙ₋₁)
X=Cl, Br
R′=alkyl, such as for example methyl, ethyl,
n-propyl, i-propyl, butyl
R′=cycloalkyl
n=1-20

e) halogeno-organosilanes of the type X(R′)₂Si(CₙH₂ₙ₊₁) and X(R′)₂Si(CₙH₂ₙ₋₁)
X=Cl, Br
R′=alkyl, such as for example methyl, ethyl, n-propyl, i-propyl, butyl
R′=cycloalkyl
n=1-20

f) cyclic polysiloxanes of the type D 3, D 4, D 5, wherein
D 3, D 4 and D 5 are understood to represent cyclic polysiloxanes with 3, 4 or 5 units of the type —O—Si(CH₃)₂—
for example octamethylcyclotetrasiloxane=D 4

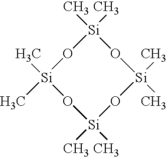

g) polysiloxanes and silicone oils of the type 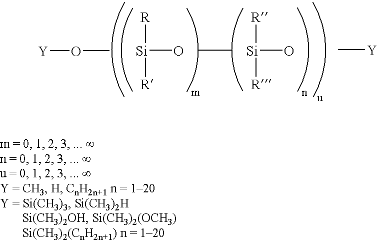
R=alkyl, such as CₙH₂ₙ₊₁, wherein n=1 to 20, aryl, such as phenyl and substituted phenyl groups, H
R′=alkyl, such as CₙH₂ₙ₊₁, wherein n=1 to 20, aryl, such as phenyl and substituted phenyl groups, H
R″=alkyl, such as CₙH_{2nα1}, wherein n=1 to 20, aryl, such as phenyl and substituted phenyl groups, H
R′″=alkyl, such as CₙH₂ₙ₊₁, wherein n=1 to 20, aryl, such as phenyl and substituted phenyl groups, H.

The following reagents can preferably be used as surface modification agents: propyltrimethoxysilane, propyltriethoxysilane, octyltrimethoxysilane (OCTMO), octyltriethoxysilane, hexadecyltrimethoxysilane, hexadecyltriethoxysilane, dimethylpolysiloxane.

Octyltrimethoxysilane and octyltriethoxysilane can be particularly preferably used.

The pyrogenically prepared, surface modified, structurally modified titanium dioxides according to the invention can be used to produce sunscreen formulations.

The invention also provides sunscreen formulations characterised in that they contain pyrogenically prepared, surface-modified, structurally modified titanium dioxides or pyrogenically prepared, surface-modified, structurally modified titanium dioxide mixed oxides.

The sunscreen formulations in the present invention may preferably also contain, in addition to one or more oil phases, one or more aqueous phases and are present, for example, in the form of W/O, O/W, W/O/W or O/W/O emulsions. Such formulations may also advantageously be microemulsions, sticks, foams (so-called mousses), solid emulsions (i.e. emulsions which are stabilised by solids, e.g. Pickering emulsions), sprayable emulsions or hydrodispersions. Furthermore, the preparations may also advantageously be oil-free and/or aqueous/alcoholic solutions.

(Macroscopic) two-phase or multi-phase systems are also advantageous according to the invention. “Two-phase or multi-phase” in the context of the present invention means that two or more phases are present in separate layers, on top of each other. It is especially advantageous in the context of the present invention if at least one of the macroscopically visible phases is a (W/O, O/W, micro) emulsion. The emulsion is perceived as one phase in this (macroscopic) consideration, although naturally a person skilled in the art is well aware that emulsions per se are formed from two or more phases homogenised with each other. The “emulsion phase” is stable over the long term so that even over a relatively long period (months, years), there is no demixing or phase separation visible within the emulsion.

The macroscopically visible phases or layers can advantageously be emulsified for the short term, for example by shaking, to give a homogeneous emulsion which is not, however, stable over the long term; rather, it demixes again over a period of minutes, hours or days to give two or more phases present in layers on top of each other.

It is particularly advantageous in the context of the present invention if at least one of the macroscopically visible phases is a microemulsion and at least one other of the macroscopically visible phases is an oil phase.

Sprayable Emulsions, In Particular Microemulsions

Sprayable O/W emulsions, in particular O/W microemulsions are particularly advantageous in the context of the present invention.

The droplet diameters of the usually “simple”, that is non-multiple, emulsions are within the range from about 1 μm up to about 50 μm. Such “macroemulsions” are, without any further colouring additives, a milky-white colour and opaque. Finer “macroemulsions”, the droplet diameter of which is within the range from about 0.5 μm to about 1 μm are, again without colouring additives, a bluey-white colour and opaque. Such “macroemulsions” usually have a high viscosity.

The droplet diameter of microemulsions in the context of the present invention, however, is within the range from about 50 to about 500 nm. These types of microemulsions have a bluey-white to translucent colour and mostly have a low viscosity. The viscosity of many microemulsions of the O/W type is comparable to that of water.

The advantage of microemulsions is that active substances present in the dispersed phase can be dispersed substantially more finely than in the dispersed phase of “macroemulsions”. Another advantage is that they are sprayable, due to their low viscosity. If microemulsions are used as cosmetics, the corresponding products are characterised by a high degree of cosmetic elegance.

O/W microemulsions which are particularly advantageous according to the invention are those which are obtainable with the help of so-called phase inversion temperature technology and which contain at least one emulsifier (emulsifier A) chosen from the group of emulsifiers with the following properties:
the lipophilicity depends on the temperature, in such a way that the lipophilicity increases when the temperature is raised and
the lipophilicity of the emulsifier decreases when the temperature is lowered.

Advantageous emulsifiers A are e.g. polyethoxylated fatty acids (PEG-100 stearate, PEG-20 stearate, PEG-150 laurath, PEG-8 distearate and the like) and/or polyethoxylated fatty alcohols (cetearath-12, cetearath-20, isoceteth-20, beheneth-20, laurath-9 etc.) and/or alkylpolyglycosides (cetearyl glycosides, stearyl glycosides, palmityl glycosides etc.).

Provided the phase inversion is initiated substantially by varying the temperature, O/W emulsions, in particular O/W microemulsions can be obtained, wherein the size of the oil droplets is determined substantially by the concentration of the emulsifier(s) used, in such a way that a higher concentration of emulsifier produces smaller droplets and a lower concentration of emulsifier leads to larger droplets. The size of the droplets is generally between 20 and 500 nm.

In the context of the present invention it is optionally advantageous to use further W/O and/or O/W emulsifiers which are not covered by the definition of emulsifier A, for example in order to increase the water-resistance of preparations in accordance with the invention. In this case, for example, alkyl methicone copolyols and/or alkyl dimethicone copolyols (in particular cetyl dimethicone copolyol, lauryl methicone copolyol), W/O emulsifiers (such as e.g. sorbitan stearate, glyceryl stearate, glycerol stearate, sorbitan oleate, lecithin, glyceryl isostearate, polyglyceryl-3-oleate, polyglyceryl-3-diisostearate, PEG-7-hydrogenated castor oil, polyglyceryl-4-distearate, acrylate/C 10-30-alkyl acrylate cross polymers, sorbitan isostearate, poloxamer 101, polyglyceryl-2-dipolyhydroxystearate, polyglyceryl-3-diisostearate, polyglyceryl-4-dipolyhydroxystearate, PEG-30-dipolyhydroxystearate, diisostearoyl polyglyceryl-3-diisostearate, glycol distearate, polyglyceryl-3-dipolyhydroxystearate) and/or fatty acid esters of sulphuric or phosphoric acid (cetyl phosphate, trilaureth-4 phosphate, trioleth-8-phosphate, stearyl phosphate, cetearyl sulfate etc.) can be used.

Further advantageously sprayable O/W emulsions in the context of the present invention are low-viscosity cosmetic or dermatological hydrodispersions which contain at least one oil phase and at least one aqueous phase, wherein the preparation is stabilised by at least one gel-producer and emulsifiers do not necessarily have to be present but one or more emulsifiers may be present.

Advantageous gel-producers for these types of preparations are, for example, copolymers of C10-30 alkyl acrylates and one or more monomers of acrylic acid, methacrylic acid or their esters. The INCI name for such compounds is “acrylate/C10-30 alkyl acrylate cross polymers”. The Pemulen® types TR1, TR2 and TRZ from the Goodrich Co. (Noveon) are particularly advantageous.

Carbopols are also advantageous gel-producers for these type of preparations. Carbopols are polymers of acrylic acid, in particular also acrylate/alkyl acrylate copolymers. Advantageous carbopols are, for example, the types 907, 910, 934, 940, 941, 951, 954, 980, 981, 1342, 1382, 2984 and 5984, also the ETD types 2020, 2050 and carbopol Ultrez 10. Further advantageous gel-producers for these types of preparations are xanthan gum, cellulose derivatives and carob seed flour.

Ethoxylated fatty alcohols or ethoxylated fatty acids (in particular PEG-100 stearate, ceteareth-20) and/or other non-ionic surface-active substances may be used as possible (optional) emulsifiers.

Furthermore, very low-viscosity to sprayable emulsions may advantageously also be W/O or water-in-silicone-oil (W/S) emulsions. Particularly advantageous emulsions are W/O or W/S emulsions which contain

at least one silicone emulsifier (W/S) with a HLB value≦8 and/or at least one W/O emulsifier with a HLB value<7 and at least one O/W emulsifier with a HLB value>10.

These types of preparations also contain at least 20 wt. % of lipids, wherein the lipid phase may advantageously also contain silicone oils or may even consist entirely of such. The silicone emulsifier(s) may advantageously be chosen from the group of alkyl methicone copolyols and/or alkyl dimethicone copolyols (e.g. dimethicone copolyols, which can be obtained from Goldschmidt AG under the commercial names ABIL® B 8842, ABIL® B 8843, ABIL® B 8847, ABIL® B 8851, ABIL® B 8852, ABIL® B 8863, ABIL® B 8873 and ABIL® B 88183, cetyl dimethicone copolyol [Goldschmidt AG/ABIL® EM 90], cyclomethicone dimethicone copolyol [Goldschmidt AG/ABIL® EM 97], lauryl methicone copolyol [Dow Corning Ltd. I Dow Corninge 5200 Formulation Aid], octyl dimethicone ethoxy glucoside [Wacker]).

W/O-emulsifiers with a HLB value<7 can advantageously be chosen from the following group: sorbitan stearate, sorbitan oleate, lecithin, glyceryl lanolate, lanolin, hydrogenated castor oil, glyceryl isostearate, polyglyceryl-3-oleate, pentaerythrityl isostearate, methylglucose dioleate, methylglucose dioleate mixed with hydroxystearate and beeswax, PEG-7-hydrogenated castor oil, polyglyceryl-4-isostearate, hexyl laurate, acrylate/C₁ₒ₋₃ₒ-alkyl acrylate cross polymers, sorbitansisostearate, polyglyceryl-2-dipolyhydroxystearate, polyglyceryl-3-diisostearate, PEG-30-dipolyhydroxystearate, diisostearoyl polyglyceryl-3-diisostearate, polyglyceryl-3-dipolyhydroxystearate, polyglyceryl-4-dipolyhydroxystearate, polyglyceryl-3-dioleate.

O/W emulsifier(s) with a HLB value>10 can advantageously be chosen from the following group: glyceryl stearate mixed with ceteareth-20, ceteareth-25, ceteareth-6 mixed with stearyl alcohol, cetylstearyl alcohol mixed with PEG-40-castor oil and sodium cetylstearyl sulfate, triceteareth-4 phosphate, glyceryl stearate, sodium cetylstearyl sulfate, lecithin trilaureth-4 phosphate, laureth-4 phosphate, stearic acid, propylene glycol stearate SE, PEG-9 stearate, PEG-20 stearate, PEG-30 stearate, PEG-40 stearate, PEG-100 stearate, ceteth-2, ceteth-20, polysorbate-20, polysorbate-60, polysorbate-65, polysorbate-100, glyceryl stearate mixed with PEG-100 stearate, ceteareth-3, isostearyl glyceryl ether, cetylstearyl alcohol mixed with sodium cetylstearyl sulfate, PEG-40 stearate, glycol distearate, polyglyceryl-2-PEG-4 stearate, ceteareth-12, ceteareth-20, ceteareth-30, methylglucose sesquistearate, steareth-10, PEG-20 stearate, steareth-21, steareth-20, isosteareth-20, PEG-45/dodecylglycol copolymer, glyceryl stearate SE, ceteth-20, PEG-20 methylglucose sesquistearate, glyceryl stearate citrate, cetyl phosphate, cetearyl sulfate, sorbitan sesquioleate, triceteareth-4 phosphate, trilaureth-4 phosphate, polyglyceryl-methylglucose distearate, potassium cetyl phosphate, isosteareth-10, polyglyceryl-2 sesquiisostearate, ceteth-10, isoceteth-20, glyceryl stearate mixed with ceteareth-20, ceteareth-12, cetyl stearyl alcohol and cetyl palmitate, PEG-30 stearate, PEG-40 stearate, PEG-100 stearate.

Aqueous-alcoholic solutions are also advantageous. They may contain 0 wt. % to 90 wt. % of ethanol. Aqueous-alcoholic solutions, in the context of the present invention, may also advantageously contain solubility promoters such as e.g. PEG-40 or PEG-60 hydrogenated castor oil.

Preparations in accordance with the present invention may also advantageously be used as cosmetic or dermatological impregnation solutions, with which, in particular water-insoluble substrates, such as e.g. woven or non-woven cloths, are moistened. These types of impregnation solutions are preferably of low viscosity, in particular sprayable (such as e.g. PIT emulsions, hydrodispersions, W/O emulsions, oils, aqueous solutions etc.) and preferably have a viscosity of less than 2000 mPa s, in particular less than 1,500 mPa s (measuring instrument: Haake Viskotester VT O₂ at 25° C.). With the assistance of these, for example cosmetic sunscreen tissues, conditioning tissues and the like can be obtained, these consisting of a combination of a soft, water-insoluble material and a low-viscosity cosmetic and dermatological impregnation solution.

The preparations in accordance with the present invention may also advantageously be presented as anhydrous oils or oil-gels or pastes. Advantageous oils are e.g. synthetic, semi-synthetic or natural oils such as, for example, rapeseed oil, rice oil, avocado oil, olive oil, mineral oil, cocoglycerides, butylene glycol dicaprylate/dicaprate, C12-15 alkyl benzoate, dicaprylyl carbonate, octyldodecanol and the like. A very wide variety of waxes with a melting point>25° C. can be used as oil gel-producers. Gel-producers from the group of aerosils, alkyl galactomannanes (e.g. N-Hance AG 200 and N-Hance AG 50 from. Hercules) and polyethylene derivatives are also advantageous.

In the context of the present invention, self-foaming, foaming, post-foaming or foamable cosmetic and dermatological preparations are also particularly advantageous.

“Self-foaming”, “foaming”, “post-foaming” and “foamable” preparations are understood to be those from which in principle foams can be produced, whether it be during the production process, during use by the user or in some other way, by the introduction of one or more gases. In these types of foams, the gas bubbles (if any) are present distributed in one (or more) liquid phase(s), wherein, macroscopically, the (expanded) preparations do not necessarily have to have the appearance of a foam. (Expanded) cosmetic or dermatological preparations according to the invention (also called foams in the following, for the sake of simplicity) may be, for example, macroscopically visible dispersed systems of gases dispersed in liquids. However, the foam character may also be visible only, for example, under a (light) microscope.

In addition, foams according to the invention, in particular when the gas bubbles are too small to be detected under a light microscope, can also be recognised by the large increase in volume of the system.

It was particularly surprising, and also involves an inventive step, that, due to the use of the alpha olefin/maleic anhydride copolymers according to the invention, the introduction of gases is supported and also a stabilising and clearly increasing foam effect can be produced over a long storage time, even at elevated temperatures (e.g. 40° C.). It was particularly astonishing that the use of special surfactants becomes unnecessary. Surprisingly, the introduction of gases is increased to an extraordinary extent when compared with the prior art. Thus, for example, foam amplification with up to 100% increased volumes of gas can be produced, without having to use foaming agents such as the surfactants which are conventionally used in the prior art.

As a result it is possible to generate formulations with high gas volumes (air and/or other gases such as oxygen, carbon dioxide, nitrogen, helium, argon, etc.) which are stable over a long period of storage at high temperatures.

Therefore, the invention also provides the use of one or more alpha olefin/maleic anhydride copolymers for the foam amplification of self-foaming, foaming, post-foaming or foamable cosmetic and dermatological preparations.

In the context of the present invention, “foam amplification” is understood to mean that the introduction of gases into the foams according to the invention is increased to an extraordinary extent when compared with introduction into preparations which are otherwise identical but which do not contain any alpha olefin/maleic anhydride copolymers according to the invention. Accordingly, foams according to the invention can take up a much higher volume of gas than preparations which do not contain any alpha olefin/maleic anhydride copolymers according to the invention.

In addition “foam amplification” is also intended to mean that the stability of the expanded preparations (“foam stability”) is greatly improved when compared with preparations which are otherwise identical but which do not contain any alpha olefin/maleic anhydride copolymers according to the invention, i.e. breakdown of the foams is delayed by the use according to the invention.

In the context of the invention, these types of preparations advantageously contain an emulsifying system which comprises
A) at least one emulsifier A, chosen from the group of fully, partly or non-neutralised, branched and/or unbranched, saturated and/or unsaturated fatty acids with a chain length of 10 to 40 carbon atoms,
B) at least one emulsifier B, chosen from the group of polyethoxylated fatty acid esters with a chain length of 10 to 40 carbon atoms and with a degree of ethoxylation of 5 to 100 and
C) at least one coemulsifier C, chosen from the group of saturated and/or unsaturated, branched and/or unbranched fatty alcohols with a chain length of 10 to 40 carbon atoms.

Emulsifier(s) A are preferably chosen from the group of fatty acids which are neutralised fully or partly with conventional alkalis (such as e.g. sodium and/or potassium hydroxide, sodium and/or potassium carbonate and mono- and/or triethanolamine). Stearic acid and stearates, isostearic acid and isostearates, palmitic acid and palmitates as well as myristic acid and myristates, for example, are particularly advantageous.

Emulsifier(s) B are preferably chosen from the following group: PEG-9 stearate, PEG-8 distearate, PEG-20 stearate, PEG-8 stearate, PEG-8 oleate, PEG-25 glyceryl trioleate, PEG-40 sorbitan lanolate, PEG-15 glyceryl ricinoleate, PEG-20 glyceryl stearate, PEG-20 glyceryl isostearate, PEG-20 glyceryl oleate, PEG-20 stearate, PEG-20 methylglucose sesquistearate, PEG-30 glyceryl isostearate, PEG-20 glyceryl laurate, PEG-30 stearate, PEG-30 glyceryl stearate, PEG-40 stearate, PEG-30 glyceryl laurate, PEG-50 stearate, PEG-100 stearate, PEG-150 laurate. Polyethoxylated stearates, for example, are especially advantageous.

Coemulsifier(s) C are, according to the invention, preferably chosen from the following group: behenyl alcohol (C22H450H), cetearyl alcohol [a mixture of cetyl alcohol (C16H330H) and stearyl alcohol (C18H370H)], lanolin alcohols (crude wool grease alcohols which represent the unsaponifiable alcohol fraction of the wool grease which is obtained after saponification of the wool grease). Cetyl and cetylstearyl alcohols are particularly preferred.

According to the invention, it is advantageous to choose the ratio by weight of emulsifier A to emulsifier B to emulsifier C (A:B:C) to be a:b:c, wherein a, b and c, independently, may be rational numbers from 1 to 5, preferably from 1 to 3. A ratio by weight of about 1:1:1 is particularly preferable.

In the context of the invention, it is advantageous to choose the total amount of emulsifiers A and B and of coemulsifier C from within the range 2 to 20 wt. %, advantageously 5 to 15 wt. %, in particular 7 to 13 wt. %, each with respect to the total weight of the formulation.

In the context of the present invention, cosmetic or dermatological preparations which are stabilised only by very finely distributed solids are particularly advantageous. Such “emulsifier-free” emulsions are also known as Pickering emulsions.

In Pickering emulsions the solid substance becomes enriched at the oil/aqueous phase boundaries, in the form of a layer, and this prevents the disperse phases running into each other. In particular the surface properties of the solid particles, which should exhibit both hydrophilic and lipophilic properties, are then of substantial importance.

The stabilising solid particles may advantageously also be treated (coated) to make the surface thereof water-repellent, wherein an amphiphilic character is produced on these solid particles, or should be preserved thereon. The surface treatment procedure may comprise providing the solid particles with a thin hydrophobic or hydrophilic layer using methods known per se.

The average particle diameter of the microfine solid particles used as a stabiliser is chosen to be preferably less than 100 μm, particularly preferably less than 50 μm. It is then substantially unimportant in what shape (platelets, rods, spheres, etc.) or modification the solid particles used are present.

The microfine solid particles are preferably chosen from the group of amphiphilic metal oxide pigments. The following are particularly advantageous:
titanium dioxides (coated and uncoated): e.g. Eusolex T-2000 from Merck, titanium dioxide MT 100 Z from Tayca Corporation
zinc oxides e.g. Z-Cote and Z-Cote HP1 from BASF AG, MZ-300, MZ-500 and MZ-505M from Tayca Corporation
iron oxides

Furthermore, it is advantageous if the microfine solid particles are chosen from the following group: boron nitrides, starch derivatives (tapioca starch, sodium corn starch octynyl succinate etc.), talcum, latex particles.

According to the invention it is advantageous if the solids-stabilised emulsions contain less than 0.5 wt. % of one or more emulsifiers, or even are completely emulsifier-free.

Furthermore, in the context of the invention, preparations which are present in the form of sticks are an advantage. From a technical point of view, most stick formulations are anhydrous grease mixtures of solid or semi-solid waxes and liquid oils, wherein highly purified paraffin oils and waxes form the basic stick matrix.

Conventional basic substances for stick-shaped preparations are, for example, liquid oils (such as e.g. paraffin oils, castor oil, isopropyl myristate, C₁₂₋₁₅ alkyl benzoate), semisolid constituents (e.g. Vaseline, lanolin), solid constituents (e.g. beeswax, ceresin and microcrystalline waxes or ozokerite) and/or high-melting waxes (e.g. carnauba wax, candelilla wax). Aqueous stick-shaped preparations are also known per se, wherein these may also be present in the form of W/O emulsions.

Cosmetic or dermatological formulations according to the invention for protecting against light may also be composed in a conventional manner and be used for cosmetic or dermatological light protection, also for the treatment, conditioning and cleaning of skin and/or hair and as make-up products in decorative cosmetics.

In accordance with their structure, cosmetic or topical dermatological compositions in the context of the present invention can be used, for example, as skin protection cream, cleansing milk, day or night cream, etc. It is optionally possible, and advantageous, to use the compositions according to the invention as the basis of pharmaceutical preparations.

When used, the cosmetic and dermatological preparations are applied in adequate amounts, in the conventional manner for cosmetics, to the skin and/or the hair.

The cosmetic and dermatological preparations in accordance with the invention may contain cosmetic auxiliary substances such as are normally used in such preparations, e.g. preservatives, preservative aids, complex-producers, bactericides, perfumes, substances to inhibit or increase the foaming effect, colorants, pigments which have a colouring effect, thickeners, moistening or moisture-retaining substances, fillers which improve the feel of the skin, fats, oils, waxes or other conventional constituents for a cosmetic or dermatological formulation such as alcohols, polyols, polymers, foam stabilisers, electrolytes, organic solvents or silicone derivatives.

Advantageous preservatives, in the context of the present invention are, for example, formaldehyde-releasing compounds (such as e.g. DMDM hydantoin, which is obtainable for example from Lonza under the commercial name Glydant™), iodopropylbutyl carbamate (e.g. obtainable from Lonza under the commercial names Glycacil-L, Glycacil-S and/or from Jan Dekker under the name Dekaben LMB), parabens (i.e. alkyl esters of p-hydroxy-benzoic acid, such as methyl, ethyl, propyl- and/or butyl paraben), phenoxyethanol, ethanol, benzoic acid and the like. Normally the preservative system according to the invention also advantageously contains preservative aids such as, for example, octoxyglycerin, glycine soy oil etc.

Advantageous complex-producers, in the context of the present invention, are for example EDTA, [S,S]-ethylenediamine disuccinate (EDDS), which is obtainable for example from Octel under the commercial name Octaquest, pentasodium ethylendiaminetetramethylene phosphonate, which is obtainable from e.g. Monsanto under the commercial name Dequest 2046 and/or iminodisuccinic acid, which is obtainable, inter alia, from Bayer AG under the commercial names Iminodisuccinat VP OC 370 (approx. 30% solution) and Baypure CX 100 solid.

Particularly advantageous preparations are also obtained when antioxidants are used as additives or active substances. According to the invention, the preparations advantageously contain one or more antioxidants. Beneficial, but nevertheless optional, antioxidants which may be used are any antioxidants which are suitable for or are commonly used in cosmetic and/or dermatological applications.

Particularly advantageously, in the context of the present invention, water-soluble antioxidants may be used, such as for example vitamins, e.g. ascorbic acid, and its derivatives.

Preferred antioxidants are also vitamin E and its derivatives as well as vitamin A and its derivatives.

The amount of antioxidants (one or more compounds) in the preparations is preferably 0.001 to 30 wt. %, particularly preferably 0.05 to 20 wt. %, in particular 0.1 to 10 wt. %, with respect to the total weight of the preparation.

If vitamin E and/or its derivatives are used as the antioxidant(s), it is advantageous to choose the relevant concentrations from within the range 0.001 to 10 wt. %, with respect to the total weight of the formulation.

If vitamin A or vitamin A derivatives or carotene or its derivatives are used as the antioxidant(s), it is advantageous to choose the relevant concentrations from within the range 0.001 to 10 wt. %, with respect to the total weight of the formulation.

It is particularly advantageous when the cosmetic preparations according to the present invention contain cosmetic or dermatologically active substances, wherein preferred active substances are antioxidants which can protect the skin from oxidative effects.

Further advantageous active substances in the context of the present invention are natural active substances and/or their derivatives such as e.g. alpha-liponic acid, phytoen, D-biotin, coenzyme Q10, alpha-glucosyl rutin, camitin, carnosin, natural and/or synthetic isoflavonoids, creatine, taurine and/or beta-alanine as well as 8-hexadecene-1,16-dicarboxylic acid (dioic acid, CAS no. 20701-68-2; provisional INCI name octadecenedioic acid).

Formulations according to the invention which contain e.g. well-known antiwrinkle active substances such as flavoneglycosides (in particular alpha-glycosyl rutin), coenzyme Q10, vitamin E and/or derivatives and the like, are particularly advantageously suitable for the prophylaxis and treatment of cosmetic or dermatological skin changes, such as occur e.g. during ageing of the skin (such as for example dryness, roug/mess and the formation of dry-skin wrinkles, itching, reduced re-greasing (e.g. after washing), visible vessel dilation (telangiectases, cuperosis), slackness and the formation of creases and wrinkles, local hyperpigmentation, hypopigmentation and changes in pigmentation (e.g. age spots), increased susceptibility to mechanical stress (e.g. chapping) and the like). Furthermore they are advantageously suitable for hindering the appearance of dry or rough skin.

The aqueous phase in preparations in accordance with the present invention may advantageously contain conventional cosmetic auxiliary agents such as, for example, alcohols, in particular those with only a few carbon atoms, preferably ethanol and/or isopropanol, diols or polyols with a few carbon atoms and their ethers, preferably propylene glycol, glycerine, butylene glycol, ethylene glycol, ethylene glycol monoethyl or monobutyl ether, propylene glycol monomethyl, monoethyl or monobutyl ether, diethylene glycol monomethyl or monoethyl ether and analogous products, polymers, foam stabilisers and electrolytes and in particular one or more thickening agents which may be advantageously chosen from the group silicon dioxide, aluminium silicates, polysaccharides and their derivatives, e.g. hyaluronic acid, xanthan gum, hydroxypropylmethyl-cellulose, and particularly advantageously from the group of polyacrylates, preferably a polyacrylate from the group of so-called carbopols [from Goodrich], for example carbopols of the types 980, 981, 1382, 2984, 5984, ETD 2020, ETD 2050, Ultrez 10, either individually or as a combination.

Preparations in accordance with the present invention may also advantageously contain self-tanning substances such as, for example, dihydroxyacetone and/or melanin derivatives in concentrations of 1 wt. % up to 8 wt. %, with respect to the total weight of the preparation.

Preparations in accordance with the present invention may also advantageously contain repellents to provide protection from midges, ticks and spiders and the like. Substances which are advantageous are, for example, N,N-diethyl-3-methylbenzamide (commercial name: Metadelphene, “DEET”), dimethyl phthalate (commercial name: Palatinol M, DMP) and in particular ethyl 3-(N-n-butyl-N-acetyl-amino)-propionate (obtainable from Merck under the commercial name Insekt Repellent™ 3535). The repellents may be used either individually or as a combination.

Substances or substance mixtures known as moisturisers are those which provide cosmetic or dermatological preparations with the property of reducing the loss of moisture from the outer layers of skin (also called transepidermal water loss (TEWL)) after application to or distribution over the surface of the skin and/or which have a positive effect on hydration of the outer layers of skin.

In the context of the present invention, advantageous moisturisers are, for example, glycerine, lactic acid and/or lactates, in particular sodium lactate, butylene glycol, propylene glycol, biosaccaride gum-1, glycine soy oil, ethylhexyloxyglycerin, pyrrolidone carboxylic acid and urea. It is also of particular advantage to use polymeric moisturisers from the group of polysaccharides which are water-soluble and/or swell in the presence of water and/or can be gelled with the aid of water. Of particular advantage are, for example, hyaluronic acid, chitosan and/or a fucose-rich polysaccharide, which is recorded in Chemical Abstracts under registration number 178463-23-5 and is obtainable from SOLABIA S. A. under the name Fucogel™ 1000. Moisturisers may also advantageously be used as anti-wrinkle agents for the prophylaxis and treatment of cosmetic or dermatological skin changes such as occur e.g. during the ageing of skin.

Cosmetic or dermatological preparations according to the invention may also advantageously contain fillers, although this is not absolutely necessary, these further improving the sensory and cosmetic properties of the formulations and producing or amplifying a velvety or silky feel to the skin. In the context of the present invention, advantageous fillers are starches and starch derivatives (such as e.g. tapioca starch, distarch phosphate, aluminium or sodium starch octenyl succinate and the like), pigments which have neither a mainly UV-filtering nor colouring effect (such as e.g. boron nitride etc.) and/or Aerosil®.

The oil phase in formulations according to the invention is advantageously chosen from the group of polar oils, for example from the group of lecithins and fatty acid triglycerides, particularly the triglycerin esters of saturated and/or unsaturated, branched and/or unbranched alkanoic acids with a chain length of 8 to 24, in particular 12 to 18 carbon atoms. Fatty acid triglycerides may advantageously be chosen from the group of synthetic, semi-synthetic and natural oils such as e.g. cocoglyceride, olive oil, sunflower oil, soy oil, ground-nut oil, rapeseed oil, almond oil, palm oil, coconut oil, castor oil, wheat-germ oil, grapeseed oil, thistle oil, evening primrose oil, macadamia nut oil and the like.

Also advantageous, according to the invention, are e.g. natural waxes of animal and plant origin such as, for example, beeswax and other insect waxes as well as berry wax, shea butter and/or lanolin (wool wax).

In the context of the present invention, further polar oil components can be chosen from the group of esters of saturated and/or unsaturated, branched and/or unbranched alkanoic acids with a chain length of 3 to 30 carbon atoms and saturated and/or unsaturated, branched and/or unbranched alcohols with a chain length of 3 to 30 carbon atoms as well as from the group of esters of aromatic carboxylic acids and saturated and/or unsaturated, branched and/or unbranched alcohols with a chain length of 3 to 30 carbon atoms. Such ester oils can then advantageously be chosen from the group octyl palmitate, octyl cocoate, octyl isostearate, octyldodecyl myristate, octyl dodecanol, cetearyl isononanoate, isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, stearyl heptanoate, oleyl oleate, oleyl erucate, erucyl oleate, erucyl erucate, tridecyl stearate, tridecyl trimellitate, as well as synthetic, semi-synthetic and natural mixtures of esters such as e.g. jojoba oil.

The oil phase may also advantageously be chosen from the group of dialkyl ethers and dialkyl carbonates, with e.g. dicaprylyl ether (Cetiol OE) and/or dicaprylyl carbonate, obtainable for example from Cognis under the commercial name Cetiol CC, being preferred.

Preferred oil component(s) are also those from the group isoeicosan, neopentyl glycol diheptanoate, propylene glycol dicaprylate/dicaprate, caprylic/capric/diglyceryl succinate, butylene glycol dicaprylate/dicaprate, C12-13-alkyl lactate, di-C12-13-alkyl tartrate, triisostearin, dipentaerythrityl hexacaprylate/hexacaprate, propylene glycol monoisostearate, tricaprylin, dimethyl isosorbide. It is particularly advantageous when the oil phase in formulations according to the invention has a concentration of C12-15-alkyl benzoate or consists entirely of this.

Advantageous oil components are also e.g. butyloctyl salicylate (obtainable for example from CP Hall under the commercial name Hallbrite BHB), hexadecyl benzoate and butyloctyl benzoate and mixtures of these (Hallstar AB) and/or diethylhexyl naphthalate (Hallbrite TQ or Corapan TQ from H&R).

Any blends of such oil and wax components are also advantageous for use in the context of the present invention.

Furthermore, the oil phase may also advantageously contain non-polar oils, for example those which are chosen from the group of branched and unbranched hydrocarbons and waxes, in particular mineral oil, Vaseline (petroleum jelly), paraffin oil, squalane and squalene, polyolefins, hydrogenated polyisobutenes and isohexadecane. Polydecenes are the preferred substances from among the polyolefins.

The oil phase may also advantageously contain a concentration of cyclic or linear silicone oils or consist entirely of such oils, wherein however it is preferred that an additional concentration of other oil components be used apart from the silicone oil(s).

Silicone oils are high molecular weight synthetic polymeric compounds in which silicon atoms are linked, via oxygen atoms, to form chains or a network and the remaining valencies of the silicon are saturated by hydrocarbon groups (generally methyl, rarely ethyl, propyl, phenyl groups etc.). Silicone oils are known systematically as polyorganosiloxanes. Methyl-substituted polyorganosiloxanes, which are by far the most important compounds in this group and are characterised by the following structural formula 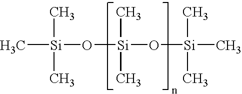
are also called polydimethylsiloxane or dimethicone (INCI). Dimethicone may have a variety of chain lengths and a variety of molecular weights.

Particularly advantageous polyorganosiloxanes in the context of the present invention are, for example, dimethylpolysiloxanes [poly(dimethylsiloxane)] which are obtainable for example from Th. Goldschmidt under the commercial names Abil 10 to 10 000. Also advantageous are phenylmethylpolysiloxanes (INCI: phenyl dimethicone, phenyl trimethicone), cyclic silicones (octamethylcyclo-tetrasiloxane and decamethylcyclopentasiloxane), which are also called cyclomethicones according to the INCI, amino-modified silicone (INCI: amodimethicone) and silicone waxes, e.g. polysiloxane-polyalkylene copolymers (INCI: stearyl dimethicone and cetyl dimethicone) and dialkoxydimethylpolysiloxanes (stearoxy dimethicone and behenoxy stearyl dimethicone), which are obtainable from Th. Goldschmidt as various types of Abil wax. However, other silicone oils are also advantageous for use in the context of the present invention, for example cetyl dimethicone, hexamethylcyclotrisiloxane, polydimethylsiloxane, poly(methylphenylsiloxane).

Preparations in accordance with the present invention may also advantageously contain one or more substances from the following group of siloxane elastomers, in order, for example, to increase the water-resistance and/or the light protection factor of the products:
a) siloxane elastomers which contain the units R₂SiO and RSiO_{1.5} and/or R₃SiO_{0.5} and/or SiO₂, wherein the individual groups R each, independently, represent hydrogen, C₁₋₂₄-alkyl (such as for example methyl, ethyl, propyl) or aryl (such as for example phenyl or toluyl), alkenyl (such as for example vinyl) and the ratio by weight of the units R₂SiO to RSiO_{1.5} is chosen to be in the range 1:1 to 30:1;
b) siloxane elastomers which are insoluble in and do not swell in silicone oil, which are obtainable by the addition reaction of an organopolysiloxane (1) which contains silicon-bonded hydrogen with an organopolysiloxane (2) which contains unsaturated aliphatic groups, wherein the proportions by weight used are chosen in such a way that the amount of hydrogen in organopolysiloxane (1) or of the unsaturated aliphatic groups in organopolysiloxans (2)
   is in the range 1 to 20 mol. % when the organopolysiloxane is not cyclic and
   is in the range 1 to 50 mol. % when the organopolysiloxane is cyclic.

In the context of the present invention, the siloxane elastomers are advantageously in the form of spherical powders or in the form of gels.

Siloxane elastomers present in the form of spherical powders and which are of advantage according to the invention are those with the INCI name dimethicone/vinyl dimethicone cross polymer, obtainable for example from DOW CORNING under the commercial name DOW CORNING 9506 Powder.

It is particularly preferred that the siloxane elastomer be used in combination with oils from hydrocarbons of animal and/or plant origin, synthetic oils, synthetic esters, synthetic ethers or mixtures of these.

It is very particularly preferred that the siloxane elastomer be used in combination with unbranched silicone oils which are liquid or pasty at room temperature or cyclic silicone oils or mixtures of these. Particularly advantageous are organopolysiloxane elastomers with the INCI name dimethicone/polysilicone-11, very particularly those Gransil types GCM, GCM-5, DMG-6, CSE Gel, PM-Gel, LTX, ININ Gel, AM-18 Gel and/or DMCM-5 which are obtainable from Grant Industries Inc.

It is very exceptionally preferred that the siloxane elastomer be used in the form of a gel made from siloxane elastomer and a lipid phase, wherein the concentration of siloxane elastomer in the gel is 1 to 80 wt. %, preferably 0.1 to 60 wt. %, each with respect to the total weight of the gel.

In the context of the present invention it is advantageous to choose the total amount of siloxane elastomers (active content) to be in the range 0.01 to 10 wt. %, advantageously 0.1 to 5 wt. %, each with respect to the total weight of the formulation. Cosmetic and dermatological preparations in accordance with the invention may contain colorants and/or coloured pigments, in particular when they are presented in the form of decorative cosmetics. The colorants and coloured pigments may be selected from the appropriate positive list in the cosmetic regulations or from the EC list of cosmetic colorants. In most cases they are identical to the colorants permitted for use in foodstuffs. Advantageous coloured pigments are, for example, titanium dioxide, mica, iron oxides (e.g. Fe₂O₃, Fe₃O₄, FeO(OH)) and/or tin oxide. Advantageous colorants are for example carmine, Berlin blue, chromium oxide green, ultramarine blue and/or manganese violet. It is particularly advantageous to choose colorants and/or coloured pigments from the Rowe Colour Index, 3rd edition, Society of Dyers and Colourists, Bradford, England, 1971.

If formulations in accordance with the invention are presented in the form of products which are applied to the face, then it is beneficial to choose one or more substances from the following group as colorants: 2,4-dihydroxyazobenzene, 1-(2′-chloro-4′-nitro-1′-phenylazo)-2-hydroxynaphthaline, Ceres red, 2-(sulfo-1-naphthylazo)-1-naphthol-4-sulfonic acid, the calcium salt of 2-hydroxy-1,2′-azonaphthaline-1′-sulfonic acid, the calcium and barium salts of 1-(2-sulfo-4-methyl-1-phenylazo)-2-naphthyl carboxylic acid, the calcium salt of 1-(2-sulfo-1-naphthylazo)-2-hydroxynaphthaline-3-carboxylic acid, the aluminium salt of 1-(4-sulfo-1-phenylazo)-2-naphthol-6-sulfonic acid, the aluminium salt of 1-(4-sulfo-1-naphthylazo)-2-naphthol-3,6-disulfonic acid, 1-(4-sulfo-1-naphthylazo)-2-naphthol-6,8-disulfonic acid, the aluminium salt of 4-(4-sulfo-1-phenylazo)-1-(4-sulfophenyl)-5-hydroxy-pyrazolone-3-carboxylic acid, the aluminium and zirconium salts of 4,5-dibromofluorescein, the aluminium and zirconium salts of 2,4,5,7-tetrabromofluorescein, 3′,4′,5′,6′-tetrachloro-2,4,5,7-tetrabromofluorescein and its aluminium salt, the aluminium salt of 2,4,5,7-tetraiodofluorescein, the aluminium salt of quinophthalone-disulfonic acid, the aluminium salt of indigo-disulfonic acid, red and black iron oxide (CIN: 77 491 (red) und 77 499 (black)), iron oxide hydrate (CIN: 77 492), manganese ammonium diphosphate and titanium dioxide.

Oil-soluble natural coloured substances such as e.g. paprika extracts, beta-carotene or cochineal are also advantageous.

In the context of the present invention, formulations with a concentration of pearlescent pigments are also advantageous. In particular, the types of pearlescent pigments listed below are preferred:
1. Natural pearlescent pigments such as e.g.
   “fish silver” (guanine/hypoxanthin mixed crystals from fish scales) and
   “mother-of-pearl” (milled mussel shells)
2. Monocrystalline pearlescent pigments such as e.g. bismuth oxychloride (BiOCl)
3. Laminated-substrate pigments: e.g. mica/metal oxide

The bases for pearlescent pigments are, for example, powdered pigments or castor oil dispersions of bismuth oxychloride and/or titanium dioxide as well as bismuth oxychloride and/or titanium dioxide on mica. The glossy pigment listed under CIN 77163, for example, is particularly advantageous.

The following types of pearlescent pigments based on mica/metal oxide, for example, are also advantageous:

| | Coating/layer | |
|---|---|---|
| Group | thickness | Colour |
| Silver-white pearlescent | TiO₂: 40-60 nm | silver |
| pigments | | |
| Interference pigments | TiO₂: 60-80 nm | yellow |
| | TiO₂: 80-100 nm | red |
| | TiO₂: 120-160 nm | green |
| Coloured glossy | Fe₂O₃ | bronze |
| pigments | Fe₂O₃ | copper |
| | Fe₂O₃ | red |
| | Fe₂O₃ | red-violet |
| | Fe₂O₃ | red-green |
| | Fe₂O₃ | black |
| Combination pigments | TiO₂/Fe₂O₃ | gold tones |
| | TiO₂/Cr₂O₃ | green |
| | TiO₂/Berlin blue | deep blue |
| | TiO₂/carmine | red |

The pearlescent pigments obtainable from e.g. Merck under the commercial names Timiron, Colorona or Dichrona are particularly preferred.

Obviously, the list of pearlescent pigments mentioned is not intended to be limiting. In the context of the present invention, advantageous pearlescent pigments can be obtained in a large number of ways known per se. For example, substrates other than mica can be coated with other metal oxides, such as e.g. silica and the like. For example, SiO₂ particles(“ronaspheres”) coated with TiO₂ and Fe₂O₃ are advantageous, are sold by Merck and are especially suitable for the optical reduction of fine wrinkles.

In addition, it may also be of advantage to entirely omit any substrate such as mica. Iron pearlescent pigments which are prepared without the use of mica are particularly preferred. Such pigments are obtainable e.g. from BASF under the commercial name Sicopearl Kupfer 1000.

Effect pigments which are obtainable from Flora Tech under the commercial name Metasomes standard/glitter and in various colours (yellow, red, green, blue) are particularly advantageous. In this case, the glitter particles are present mixed with various auxiliary substances and colorants (such as for example the colorants with the Colour Index (CI) numbers 19140, 77007, 77289, 77491).

The colorants and pigments may be present either individually or as a mixture and may be coated onto each other, wherein different colour effects can be produced in general by different coating thicknesses. The total amount of colorants and colour-providing pigments is advantageously chosen to be e.g. in the range 0.1 wt. % to 30 wt. %, preferably 0.5 to 15 wt. %, in particular 1.0 to 10 wt. %, each with respect to the total weight of the preparations.

It is also advantageous, in the context of the present invention, to produce cosmetic and dermatological preparations, the main purpose of which is not to protect from sunlight but which nevertheless do contain a concentration of further UV-protective substances. Thus, for example, UV-A or UV-B filter substances are usually incorporated into day creams or make-up products. Anti-UV substances, like antioxidants and, if desired preservatives, provide active protection to the preparation itself against spoilage. Cosmetic and dermatological preparations which are presented in the form of a sunscreen are also beneficial.

Accordingly, in the context of the present invention, the preparations preferably contain at least one further UV-A, UV-B and/or broad band filter substance. The formulations may optionally also contain, although this is not necessary, one or more organic and/or inorganic pigments as UV filter substances which may be present in the aqueous and/or oil phase.

Furthermore, preparations in accordance with the present invention may also advantageously be present in the form of so-called oil-free cosmetic or dermatological emulsions which contain an aqueous phase and at least one UV filter substance which is liquid at room temperature as a further phase and which may particularly advantageously also be free of further oil components.

In the context of the present invention, particularly advantageous UV filter substances which are liquid at room temperature are homomenthyl salicylate (INCI: homosalate), 2-ethylhexyl-2-cyano-3,3-diphenylacrylate (INCI: octocrylene), 2-ethylhexyl-2-hydroxybenzoate (2-ethylhexyl salicylate, octyl salicylate, INCI: ethylhexyl salicylate) and esters of cinnamic acid, preferably the (2-ethylhexyl) ester of 4-methoxycinnamic acid (2-ethylhexyl-4-methoxycinnamate, INCI: ethylhexyl methoxycinnamate) and the isopentyl ester of 4-methoxycinnamic acid (isopentyl-4-methoxycinnamate, INCI: isoamyl p-methoxycinnamate), 3-(4-(2,2-bis-ethoxycarbonylvinyl)-phenoxy)propenyl)-methoxysiloxane/dimethylsiloxane copolymer which is obtainable, for example, from Hoffmann La Roche under the commercial name Parsol® SLX.

Preferred inorganic pigments are metal oxides and/or other metal compounds which are insoluble or barely soluble in water, in particular the oxides of titanium (TiO₂), zinc (ZnO), iron (e.g. Fe₂O₃), zirconium (ZrO₂), silicon (SiO₂), manganese (e.g. MnO), aluminium (Al₂O₃), cerium (e.g. Ce₂O₃), mixed oxides of the corresponding metals and blends of such oxides as well as barium sulfate (BaSO₄).

In the context of the invention, the pigments may advantageously also be used in the form of commercially available oily or aqueous predispersions. Dispersion aids and/or solubility promoters may advantageously be added to these predispersions.

According to the invention, the pigments may advantageously be surface-treated (“coated”), wherein, for example, a hydrophilic, amphiphilic or hydrophobic character may be produced or preserved. This surface treatment may comprise providing the pigments with a thin hydrophilic and/or hydrophobic inorganic and/or organic layer using a process known per se. In the context of the present invention, the various coatings may also contain water.

Inorganic surface coatings, in the context of the present invention, may consist of aluminium oxide (Al₂O₃), aluminium hydroxide Al(OH)₃, or aluminium oxide hydrate (also: alumina, CAS-No.: 1333-84-2), sodium hexametaphosphate (NaPO₃)₆, sodium metaphosphate (NaPO₃)ₙ, silicon dioxide (SiO₂) (also: silica, CAS-No.: 7631-86-9), or iron oxide (Fe₂O₃). These inorganic surface coatings may be present on their own, in combination with each other and/or in combination with organic coating materials.

Organic surface coatings, in the context of the present invention, may consist of vegetable or animal aluminium stearate, vegetable or animal stearic acid, lauric acid, dimethylpolysiloxane (also: dimethicone), methylpolysiloxane (methicone), simethicone (a mixture of dimethylpolysiloxane with an average chain length of 200 to 350 dimethylsiloxane-units and silica gel) or alginic acid. These organic surface coatings may be present on their own, in combination with each other and/or in combination with inorganic coating materials. Zinc oxide particles and predispersions of zinc oxide particles which are suitable according to the invention are obtainable under the following commercial names and from the companies listed below:

| Commercial name | Coating | Manufacturer |
|---|---|---|
| Z-Cote HP 1 | 2% Dimethicone | BASF |
| Z-Cote | / | BASF |
| ZnO NDM | 5% Dimethicone | H & R |
| MZ-303S | 3% Methicone | Tayca Corporation |
| MZ-505S | 5% Methicone | Tayca Corporation |

Titanium dioxide particles and predispersions of titanium dioxide particles which are suitable are obtainable under the following commercial names and from the companies listed below:

| Commercial name | Coating | Manufacturer |
|---|---|---|
| MT-100TV | Aluminium hydroxide/ | Tayca Corporation |
| | Stearic acid | |
| MT-100Z | Aluminium hydroxide/ | Tayca Corporation |
| | Stearic acid | |
| Eusolex T-2000 | Alumina/Simethicone | Merck KgaA |
| Titanium dioxide T805 | Octyltrimethoxysilane | Degussa |
| (Uvinul TiO₂) | | |
| Tioveil AQ 10PG | Alumina/Silica | Solaveil/Uniquema |
| Eurolex T-aqua | Water/Alumina/Sodium | Merck |
| | metaphosphate | |

Other advantageous pigments are latex particles. Latex particles which are suitable according to the invention are described in the following documents: U.S. Pat. No. 5,663,213 and EP 0 761 201. Particularly advantageous latex particles are those which are formed from water and styrene/acrylate copolymers and are obtainable e.g. from Rohm & Haas under the commercial name “Alliance SunSphere”.

Advantageous UV-A filter substances, in the context of the present invention, are dibenzoylmethane derivatives, in particular 4-(tert.-butyl)-4′-methoxydibenzoylmethane (CAS-No. 70356-09-1), which is sold by Givaudan under the marque Parsol™ 1789 and by Merck under the commercial name Eusolex™ 9020.

Other advantageous UV filter substances, in the context of the invention, are sulfonated, water-soluble UV filters, such as e.g.:
phenylene-1,4-bis-(2-benzimidazyl)-3,3′-5,5′-tetrasulfonic acid and its salts, especially the corresponding sodium, potassium or triethanolammonium salts, in particular the disodium salt of phenylene-1,4-bis-(2-benzimidazyl)-3,3′-5,5′-tetrasulfonic acid with the INCI name disodium phenyl dibenzimidazol tetrasulfonate (CAS-No.: 180898-37-7), which is obtainable, for example, from Haarmann & Reimer under the commercial name Neo Heliopan AP;
salts of 2-phenylbenzimidazol-5-sulfonic acid, such as its sodium, potassium or triethanolammonium salt as well as the sulfonic acid itself with the INCI name phenylbenzimidazole sulfonic acid (CAS.-No. 27503-81-7), which is obtainable, for example, from Merck under the commercial name Eusolex 232 or from Haarmann & Reimer under the name Neo Heliopan Hydro;
1,4-di(2-oxo-10-sulfo-3-bomylidenemethyl)-benzene (also: 3,3′-(1,4-phenylenedimethylene)-bis-(7,7-dimethyl-2-oxo-bicyclo-[2.2.1]hept-1-ylmethane sulfonic acid) and its salts (particularly the corresponding 10-sulfato compounds, in particular the corresponding sodium, potassium or triethanolammonium salt), which is also called benzene-1,4-di(2-oxo-3-bomylidenemethyl-10-sulfonic acid). Benzene-1,4-di(2-oxo-3-bomylidenemethyl-10-sulfonic acid) has the INCI name terephthalidene dicampher sulfonic acid (CAS.-No.: 90457-82-2) and is obtainable, for example, from Chimex under the commercial name Mexoryl SX;
sulfonic acid derivatives of 3-benzylidenecampher, such as e.g. 4-(2-oxo-3-bornylidenemethyl)benzenesulfonic acid, 2-methyl-5-(2-oxo-3-bomylidenemethyl)sulfonic acid and their salts.

Advantageous UV filter substances, in the context of the present invention, are also benzoxazole derivatives which are characterised by the following structural formula, 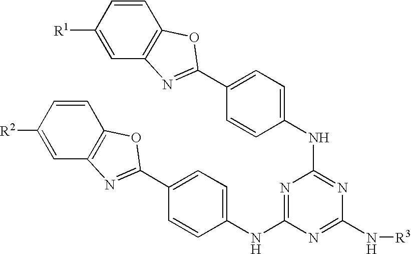
wherein R¹, R² and R³, independently, are chosen from the group of branched or unbranched, saturated or unsaturated alkyl groups with 1 to 10 carbon atoms. According to the invention, it is particularly advantageous that the groups R¹ and R² are chosen to be identical and are in particular from the group of branched alkyl groups with 3 to 5 carbon atoms. It is also particularly advantageous, in the context of the present invention, that R³ be an unbranched or branched alkyl group with 8 carbon atoms, in particular the 2-ethylhexyl group.

A particularly preferred bezoxazole derivative, according to the invention, is 2,4-bis-[5-1 (dimethylpropyl)-benzoxazol-2-yl-(4-phenyl)-imino]-6-(2-ethylhexyl)-imino-1,3,5-triazine with the CAS No. 288254-16-0, which is characterised by the structural formula given 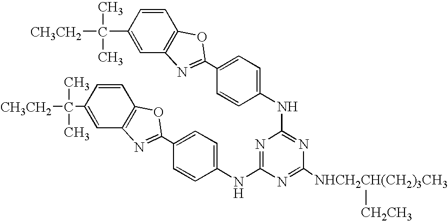
below and is obtainable from 3V Sigma under the commercial name Uvasorb™ K2A. The benzoxazole derivative(s) are advantageously present in dissolved form in cosmetic preparations according to the invention. It may also be of advantage when the benzoxazole derivative(s) are present in pigmenting, i.e. undissolved, form, for example with particle sizes from 10 nm up to 300 nm.

Advantageous UV filter substances, in the context of the present invention, are also so-called hydroxybenzophenones. Hydroxybenzophenones are characterized by the structural formula given below: 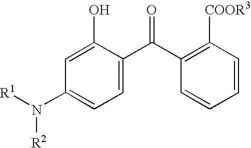
in which
R¹ and R², independently, represent hydrogen, C₁-C₂₀-alkyl, C₃-C₁₀-cycloalkyl or C₃-C₁₀-cycloalkenyl, wherein the substituents R¹ and R², together with the nitrogen atom to which they are bonded, may form a 5- or 6-membered ring and
R³ represents a C₁-C₂₀-alkyl group.

A particularly advantageous hydroxybenzophenone, in the context of the present invention, is hexyl 2-(4′-diethylamino-2′-hydoxybenzoyl)-benzoate (also: aminobenzophenone), which is characterised by the structural formula given below: 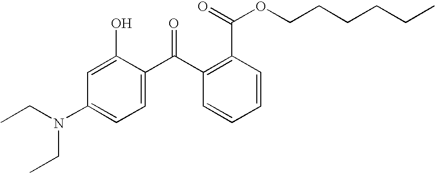 and is obtainable from BASF under the name Uvinul A Plus.

Advantageous UV filter substances, in the context of the present invention, are also so-called broad band filters i.e. filter substances which absorb both UV-A and UV-B radiation.

Advantageous broad band filters or UV-B filter substances are, for example, triazine derivatives such as e.g.
2,4-bis-[4-(2-ethylhexyloxy)-2-hydroxy]-phenyl-6-(4-methoxypheny( )-1,3,5-triazine (INCI: bis-ethylhexyloxylphenol methoxyphenyl triazine), which is obtainable from CIBA-Chemikalien GmbH under the commercial name Tinosorb™ S;
dioctylbutylamidotriazone (INCI: diethylhexyl butamido triazone), which is obtainable from Sigma 3 V under the commercial name UVASORB HEB;
the tris(2-ethylhexyl) ester of 4,4′,4″-(1,3,5-triazine-2,4,6-triyltrümino)-tris-benzoic acid, also: 2,4,6-tris-[anilino-(p-carbo-2′-ethyl-1′-hexyloxy)]-1,3,5-triazine (INCI: ethylhexyl triazone), which is sold by BASF Aktiengesellschaft under the commercial name UVINUL™ T 150;
2-[4,6-bis(2,4-dimethylphenyl)-1,3,5-triazine-2-yl]-5-(octyloxy)phenol (CAS No.: 2725-22-6).

An advantageous broad band filter, in the context of the present invention, is also 2,2-methylene-bis-(6-(2H-benzotriazol-2-yl)-4-(1,1,3,3-tetramethylbutyl)-phenol) (INCI: methylene bis-benztriazolyl tetramethylbutylphenol), which is obtainable e.g. from CIBA-Chemikalien GmbH under the commercial name Tinosorb™ M.

An advantageous broad band filter, in the context of the invention, is also 2-(2H-benzotriazol-2-yl)-4-methyl-6-[2-methyl-3-[1,3,3,3-tetramethyl-1-[(trimethylsilyl)oxy]disiloxanyl]propyl]-phenol (CAS-No.: 155633-54-8) with the INCI name drometrizole trisiloxane.

Other UV filter substances may be oil-soluble or water-soluble. Advantageous oil-soluble filter substances are, for example:
3-benzylidenecampher derivatives, preferably 3-(4-methylbenzylidene)campher, 3-benzylidenecampher;
4-aminobenzoic acid derivatives, preferably 2-ethylhexyl 4-(dimethylamino)-benzoate, amyl 4-(dimethylamino)-benzoate;
2,4,6-trianilino-(p-carbo-2′-ethyl-1′-hexyloxy)-1,3,5-triazine;
esters of benzalmalonic acid, preferably di-2-ethylhexyl 4-methoxybenzalmalonate;
esters of cinnamic acid, preferably 2-ethylhexyl 4-methoxycinnamate, isopentyl 4-methoxycinnamate;
derivatives of benzophenone, preferably 2-hydroxy-4-methoxybenzophenone, 2-hydroxy-4-methoxy-4′-methylbenzophenone, 2,2′-dihydroxy-4-methoxybenzophenone and
UV filters bonded to polymers.

Advantageous water-soluble filter substances are, for example: sulfonic acid derivatives of 3-benzylidenecampher, such as e.g. 4-(2-oxo-3-bomylidenemethyl)-benzenesulfonic acid 2-methyl-5-(2-oxo-3-bomylidenemethyl)sulfonic acid and their salts. Another light-protective filter substance which is advantageously used according to the invention is ethylhexyl-2-cyano-3,3-diphenylacrylate (octocrylene), which is obtainable from BASF under the name Uvinul® N 539 T.

Particularly advantageous preparations, in the context of the present invention, which are characterised by high or very high UV-A protection preferably also contain, in addition to the filter substance(s) according to the invention, other UV-A and/or broad band filters, in particular dibenzoylmethane derivatives [for example 4-(tert.-butyl)-4′-methoxydibenzoylmethane] and/or das 2,4-bis-[4-(2-ethylhexyloxy)-2-hydroxy]-phenyl-6-(4-methoxyphenyl)-1,3,5-triazine and/or the disodium salt of phenylene-1,4-bis-(2-benzimidazyl)-3,3′-5,5′-tetrasulfonic acid, each being used individually or in any combination with each other.

The lists of UV filters mentioned, which can be used in the context of the present invention, are obviously not intended to be limiting.

Preparations in accordance with the present invention advantageously contain substances which absorb UV radiation in the UV-A and/or UV-B region in a total amount of e.g. 0.1 wt. % to 30 wt. %, preferably 0.5 to 20 wt. %, in particular 1.0 to 15.0 wt. %, each with respect to the total weight of the preparations, in order to provide cosmetic preparations which protect the hair or the skin from the entire range of ultraviolet radiation.

Preparations in the context of the invention may advantageously also contain other substances which increase the water-resistance of the products. Polyoxyethylene-polyoxypropylene block polymers (CTFA-name: polaxamers, CAS-No. 9003-11-6) which are soluble in or dispersible in water and have the structure given below are advantageous:

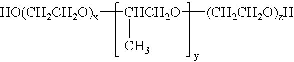

wherein x, y and z represent integers from within the range 2 to 130, in particular 15 to 100 and x and z are identical, but the value chosen depends on the value of y.

From among these compounds, the following are advantageously used: in particular polaxamer 188 [where x=75, y=30 and z=75], which can be purchased from BASF under the commercial name Lutrol F 68 (previously: Pluronic F 68), polaxamer 185 [where x=19, y=30 and z=19] (Lubrajel WA from ISP), polaxamer 235 [where x=27, y=39 and z=27 ] (Pluronic F 85 from BASF) and/or polaxamer 238 [where x=97, y=39 and z=97] (Pluronic F 88 from BASF).

Other advantageous substances which can contribute to increasing water-resistance, but are incorporated into the oil phase of the preparations in accordance with the present invention, are certain wax components, such as acetylated glycol stearate with tristearin (e.g. Unitwix from ISP with the INCI name: acetylated glycol stearate and tristearin), C18-36 fatty acid triglyceride (e.g. Syncrowax HGLC from Crode GmbH with the INCI name: C18-36 acid triglyceride) and also the substance obtainable from New Phase Technologies under the commercial name “Perfroma V 825” (a synthetic wax) as well as the use of PEG-45 dodecyl glycol copolymer (INCI: PEG-45 dodecyl glycol copolymer), PEG-22 dodecyl glycol copolymer (INCI: PEG-22 dodecyl glycol copolymer), methoxy PEG-22 dodecyl glycol copolymer (INCI: methoxy PEG-22 dodecyl glycol copolymer) which are obtainable from AKZO Nobel.

It is particularly advantageous, in the context of the present invention, to combine the polymers used in accordance with the invention with one or more of the substances mentioned, in order to improve the water-resistance of the preparations still further.

The following examples are intended to explain the present invention, without restricting it. The numerical values in the examples refer to percentages by weight, with respect to the total weight of the preparations.

### EXAMPLES

1. Surface Modification and Structural Modification

The pyrogenically prepared titanium dioxides are treated with octyltrimethoxysilane and structurally modified. The details are given in Table 2.

TABLE 2
Preparation of surface-modified and structurally modified titanium dioxides

| | Surface modification | | | | | | |
|---|---|---|---|---|---|---|---|
| | | | Parts SM**/ | Conditioning | Conditioning | Structural modification | |
| | | | 100 parts | temperature | time | Structural | Post- |
| | Oxide* | SM** | oxide | [° C.] | [h] | modification | milling*** |
| Example 1 | 2 | OCTMO | 10 | 120 | 2 | yes | no |
| Example 2 | 2 | OCTMO | 10 | 120 | 2 | yes | TDM |
| Example 3 | 2 | OCTMO | 10 | 120 | 2 | yes | CAM |
| Example 4 | 2 | OCTMO | 10 | 120 | 2 | yes | CAM |
| Example 5 | 1 | OCTMO | 10 | 120 | 2 | yes | no |
| Example 6 | 1 | OCTMO | 10 | 120 | 2 | yes | TDM |
| Example 7 | 1 | OCTMO | 10 | 120 | 2 | yes | CAM |
| Example 8 | 1 | OCTMO | 10 | 120 | 2 | yes | CAM |
| Example 9 | 1 | OCTMO | 10 | 120 | 2 | yes | no |
| Example 10 | 1 | OCTMO | 10 | 120 | 2 | yes | TDM |
| Example 11 | 1 | OCTMO | 10 | 120 | 2 | yes | CAM |
| Example 12 | 1 | OCTMO | 10 | 120 | 2 | yes | CAM |
| Example 13 | 1 | OCTMO | 10 | 120 | 2 | yes | no |
| Example 14 | 1 | OCTMO | 10 | 120 | 2 | yes | TDM |
| Example 15 | 1 | OCTMO | 10 | 120 | 2 | yes | CAM |
| Example 16 | 1 | OCTMO | 10 | 120 | 2 | yes | CAM |

*Oxide 1 = Aeroxide ® TiO₂ P25; Oxide 2 = Aeroxide ® TiO₂ PF2;
**SM = Surface modification reagent
***TDM = Toothed disc mill; CAM = Compressed air mill (=octyltrimethoxysilane)

TABLE 3
Physical and chemical data for surface-modified
and structurally modified titanium dioxides

| | Compacted | | | Loss on | Loss on | BET spec. |
|---|---|---|---|---|---|---|
| | bulk density | | C-content | drying | ignition | surface area |
| | [g/l] | pH | [%] | [%] | [%] | [m²/g] |
| Example 1 | 587 | 3.3 | 3.1 | 0.5 | 4.4 | 45 |
| Example 2 | 418 | 3.4 | 3.5 | 0.2 | 6.5 | 47 |
| Example 3 | 183 | 3.2 | 3.5 | 0.5 | 4.3 | 45 |
| Example 4 | 162 | 3.2 | 3.5 | 0.3 | 4.5 | 45 |
| Example 5 | 705 | 3.2 | 3.0 | 0.3 | 3.5 | 50 |
| Example 6 | 573 | 3.3 | 2.9 | 0.2 | 3.7 | 49 |
| Example 7 | 232 | 3.2 | 2.9 | 0.4 | 3.3 | 52 |
| Example 8 | 221 | 3.1 | 3.0 | 0.4 | 3.4 | 52 |
| Example 9 | 922 | 3.1 | | 0.4 | 3.6 | 47 |
| Example 10 | 712 | 3.2 | 2.9 | 0.2 | 3.8 | 46 |
| Example 11 | 238 | 3.3 | 2.9 | 0.3 | 3.5 | 46 |
| Example 12 | 247 | 3.2 | 3.0 | 0.4 | 3.3 | 49 |
| Example 13 | 669 | 3.2 | | 0.2 | 3.4 | 53 |
| Example 14 | 504 | 3.1 | 2.8 | 0.2 | 3.6 | 52 |
| Example 15 | 241 | 3.2 | 2.8 | 0.3 | 3.4 | 49 |
| Example 16 | 206 | 3.1 | 2.9 | 0.4 | 3.3 | 50 |

2. Sunscreen Formulations

Oxides in accordance with examples 1-4 and 9-16 are dispersed and the transparency and viscosity are tested using the following methods.

Preparation of the Dispersion

278.25 g TEGOSOFT® TN are initially introduced into a 500 ml PE beaker and 21.75 g of the titanium dioxide powder to be tested is stirred in at 470 rpm, with the aid of a dissolver (Pendraulik type LM34 No. 29490, disc diameter 6 cm) and then dispersed for five minutes at 3000 rpm.

Following this, the dispersion is dispersed with an Ultra-Turrax stirrer (Polytron PT3100, dispersion accessory PT-DA 3020/2 EC) for two minutes at 15,000 rpm. Finally, the dispersion is dispersed for a further five minutes in a water-cooled container using the Ultra-Turrax stirrer at 15,000 rpm, wherein dispersion accessory PT-DA 3030-6060/3 EC is now used.

Transparency (T ΔL*)

The transparencies of the 7.25 wt. % dispersions are determined using a Spektralphotometer Data Color SF600 Plus. The dispersions are applied to lacquered black cardboard using a 12 μm spiral spreader and the Erichsen Testing Equipment K Control Coater coating instrument, at rate of coating level 2. Three measurement points were measured per coating. The mean value of these 3 measurement points was calculated. To protect the instrument, the measurements were performed using a spacer ring.

The calculation was made using the CIE-L*a*b* system, type of light D65/10°. The instrument was calibrated with a black standard BHB SF600, a hollow block and a white standard no. 3138. The ΔL* value corresponded to the brightness or transparency of the dispersion. This value is calculated from the mean value determined minus the value of the black cardboard. The L value of the lacquered black cardboard is about L*=8. The lower the ΔL* value the more transparent is the dispersion.

UV Visible Spectra (TM 320 and 380 nm)

The UV visible spectra of 3 wt. % dispersions are measured in a removable 10 μm quartz glass cell using a UV visible Spektralphotometer Specord 200 with a photometer sphere (Analytik Jena AG). For this purpose, the oily dispersions described above are diluted with Tego-soft TN. While stirring with the dissolver (Pendraulik Typ LM34 Nr.29490, disc diameter 5 cm; 1000-4000 rpm), AEROSIL® 200 is then added in portions in order to produce a gel-like material and to stabilise the oxide.

After the final addition of AEROSIL, post-dispersion must be continued for at least 2 minutes in order to ensure homogeneous distribution of the AEROSIL. The results give the transmission (%) over the range 290-500 nm.

Viscosity (V)

The viscosity is determined using a Brookfield Rheometer RVDV-III+cP. Measurement is performed in a PE mixing beaker (350 ml) using the RV spindle attachment at 10 rpm. The value is read off in mPas after 1 minute.

The results of these tests are summarized in Table 4.

TABLE 4
Characterizing surface-modified and structurally modified
pyrogenic titanium dioxides and titanium/iron mixed oxides
from the examples (9-12 and 17-24)

| | Trans- | Trans- | Trans- | Viscos- |
|---|---|---|---|---|
| | parency | mission | mission | ity |
| Name | (TΔL*) | 320 nm (%) | 380 nm (%) | (mPas) |
| Comparison example | 17 | 4 | 13 | 732 |
| AEROXIDE TiO2 T817 | | | | |
| Example 1 | 17 | 2 | 10 | 536 |
| Example 2 | 17 | 2 | 13 | 652 |
| Example 3 | 16 | 1 | 8 | 660 |
| Example 4 | 19 | 2 | 13 | 636 |
| Comparison example | 21 | 3 | 8 | 676 |
| AEROXIDE TiO2 T805 | | | | |
| Example 9 | 16 | 3 | 8 | 452 |
| Example 10 | 18 | 2 | 12 | 396 |
| Example 11 | 22 | 1 | 7 | 516 |
| Example 12 | 20 | 2 | 8 | 520 |
| Example 13 | 20 | 2 | 7 | 660 |
| Example 14 | 19 | 3 | 10 | 632 |
| Example 15 | 18 | 2 | 8 | 560 |
| Example 16 | 19 | 4 | 15 | 628 |

The advantages of the products according to the invention in accordance with examples 1-4 as compared with the comparison example AEROXIDE TiO₂ T 817 are:
low transmission and improved absorption at 320 nm
reduced thickening effect. This enables the production of highly filled dispersions.

The advantages of the products from examples 9-16 as compared with the comparison example AEROXIDE TiO₂ T 805 are:
improved transparency
partly increased transmission at 380 nm and thus a lower whitening effect

low thickening effect. This enables the production of highly filled dispersions.

Sunscreen formulations 1

| | % | Constituent | INCI |
|---|---|---|---|
| A. | 3.00 | Isopropyl myristate | Isopropyl myristate |
| | 8.00 | Jojoba oil | Simmondsia chinensis |
| | | | (Jojoba) seed oil |
| | 4.00 | Uvinul ® MC 80 | Octyl methoxycinnamate |
| | 1.00 | Abil ® 350 | Dimethicone |
| | 6.00 | Cremophor ® WO 7 | PEG-7 hydrogenated castor oil |
| | 2.00 | Ganex ® V 216 | PVP/hexadecene copolymer |
| | 2.00 | Elfacos ® ST 9 | PEG-45/dodecyl glycol copolymer |
| | 2.00 | Uvinul ® MBC 95 | 4-methylbenzylidene camphor |
| B | 3.00 | Finely divided | Titanium dioxide |
| | | titanium dioxide | (and iron oxide) |
| | 5.00 | Z-Cote ® HP 1 | Zinc oxide (and) dimethicone |
| C | 1.00 | Magnesium | Magnesium sulfate |
| | | sulfate-7-hydrate | |
| | 5.00 | Glycerin 87% | Glycerin |
| | 0.20 | Edeta ® BD | Disodium EDTA |
| | 0.30 | Germoll ® 115 | Imidazolidinyl urea |
| | 57.00 | Water deionised | Water |
| | q.s. | Perfume | |
| | 0.50 | Euxyl ® K3000 | Phenoxyethanol, Methylparaben, |
| | | | Butylparaben, Ethylparaben, |
| | | | Propylparaben, Isobutyl′ paraben |

Phase A is heated to 80° C., phase B is added and homogenised for 3 minutes.
Phase C is heated to 80° C. and stirred into the mixture of phases A and B, with homogenization.

Sunscreen formulations 2

| | % | Constituent | INCI |
|---|---|---|---|
| A. | 6.00 | Cremophor ® WO 7 | PEG-7 Hydrogenated |
| | | | Castor Oil |
| | 2.00 | Elfacos ® ST 9 | PEG-45/Dodecyl Glycol Copolymer |
| | 3.00 | Isopropyl myristate | Isopropyl Myristate |
| | 8.00 | Jojoba Oil | Jojoba (Buxus Chinensis) |
| | | | Oil |
| | 4.00 | Uvinul ® MC 80 | Octyl Methoxycinnamate |
| | 2.00 | Uvinul ® MBC 95 | 4-Methylbenzylidene |
| | | | Camphor |
| | 3.00 | Finely divided | Titanium dioxide |
| | | titanium dioxide | (and iron oxide) |
| | 1.00 | Abil ® 350 | Dimethicone |
| | 5.00 | Z-Cote ® HP 1 | Zinc oxide, dimethicone |
| B | 0.20 | Edeta ® BD | Disodium EDTA |
| | 5.00 | Glycerin 87% | Glycerin |
| | q.s. | Preservative | |
| | 60.80 | Water deionised | Aqua deionised |
| C | q.s. | Perfume | |

Phase A and B are heated, separately, to 85° C. Then phase B is stirred into phase A and homogenised. The mixture is cooled to 40° C, phase C is added and then homogenised again.

Results

| | Formulation 1 | | Formulation 2 | |
|---|---|---|---|---|
| | Trans- | Feel on | Trans- | Feel on |
| | parency | the skin | parency | the skin |
| Comparison example | satis- | satis- | satis- | satis- |
| AEROXIDE TiO2 T805 | factory | factory | factory | factory |
| Comparison example | satis- | satis- | satis- | satis- |
| AEROXIDE TiO2 T817 | factory | factory | factory | factory |
| Example 3 | good | good | good | good |
| Example 9 | good | good | good | good |
| Example 10 | good | good | good | good |
| Example 15 | good | good | good | good |

Further variations and modifications of the foregoing will be apparent to those skilled in the art and are intended to be encompassed by the claims appended hereto.

European patent application 04 030 588.0, filed Dec. 23, 2004, is relied on and incorporated herein by reference.

1. Pyrogenically prepared, surface-modified, structurally modified titanium dioxides and pyrogenically prepared, surface-modified, structurally modified titanium dioxide mixed oxides.

2. A process for preparing pyrogenically prepared, surface-modified, structurally modified titanium dioxides and pyrogenically prepared, surface-modified, structurally modified titanium dioxide mixed oxides in accordance with claim 1, characterised in that pyrogenically prepared titanium dioxide or pyrogenically prepared titanium dioxide mixed oxide are surface-modified and then structurally modified using a ball mill and optionally post-milled.

3. Use of the pyrogenically prepared, surface-modified, structurally modified titanium dioxides in accordance with claim 1 to prepare sunscreen formulations.

4. Sunscreen formulations, characterised in that they contain pyrogenically prepared, surface-modified, structurally modified titanium dioxides and pyrogenically prepared, surface-modified, structurally modified titanium dioxide mixed oxides.

5. The pyrogenically prepared, surface-modified, structurally modified titanium dioxide and pyrogenically prepared, surface modified, structurally modified titanium dioxide mixed oxide according to claim 1, wherein the titanium dioxide contains iron oxide.

6. The pyrogenically prepared, surface-modified, structurally modified titanium dioxide and pyrogenically prepared, surface modified, structurally modified titanium dioxide mixed oxide according to claim 1, wherein the titanium dioxide has been surface modified with a surface modification agent selected from the group consisting of:
a) organosilanes of the type (RO)₃Si(CₙH₂ₙ₊₁) and (RO)₃Si(CₙH₂ₙ₋₁)
R=alkyl, such as for example methyl, ethyl, n-propyl,
i-propyl, butyl-
n=1-20
b) organosilanes of the type R′ₓ(RO)_{y}Si(CₙH₂ₙ₊₁) and
R′x(RO)ySi(CₙH₂ₙ₋₁)
R=alkyl, such as for example methyl, ethyl,
n-propyl, i-propyl, butyl
R′=alkyl, such as for example methyl, ethyl,
n-propyl, i-propyl, butyl
R′=cycloalkyl
n=1-20
x+y=3
x=1,2
y=1,2
c) halogeno-organosilanes of the type X₃Si(CₙH₂ₙ₊₁) and X₃Si(CₙH₂ₙ₋₁)
X=Cl, Br
n=1-20
d) halogeno-organosilanes of the type X₂(R′)Si(CₙH₂ₙ₊₁) and X₂(R′)Si(CₙH₂ₙ₋₁)
X=Cl, Br
R′=alkyl, such as for example methyl, ethyl,
n-propyl, i-propyl, butyl
R′=cycloalkyl
n=1-20
e) halogeno-organosilanes of the type X(R′)₂Si(CₙH₂ₙ₊₁) and X(R′)₂Si(CₙH₂ₙ₋₁)
X=Cl, Br
R′=alkyl, such as for example methyl, ethyl, n-propyl, i-propyl, butyl
R′=cycloalkyl
n=1-20
f) cyclic polysiloxanes of the type D 3, D 4, D 5, wherein
D 3, D 4 and D 5 are understood to represent cyclic polysiloxanes with 3, 4 or 5 units of the type —O—Si(CH₃)₂—
for example octamethylcyclotetrasiloxane=D 4 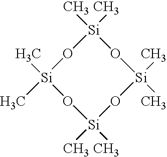
h) polysiloxanes and silicone oils of the type 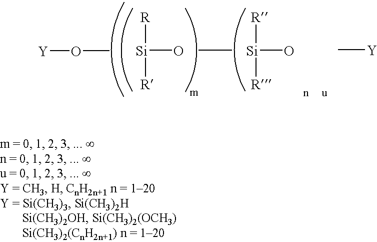
R=alkyl, such as CₙH₂ₙ₊₁, wherein n=1 to 20, aryl, such as phenyl and substituted phenyl groups, H
R′=alkyl, such as CₙH₂ₙ₊₁, wherein n=1 to 20, aryl, such as phenyl and substituted phenyl groups, H
R″=alkyl, such as CₙH₂ₙ₊₁, wherein n=1 to 20, aryl, such as phenyl and substituted phenyl groups, H
R′″=alkyl, such as CₙH₂ₙ₊₁, wherein n=1 to 20, aryl, such as phenyl and substituted phenyl groups, H.

7. The pyrogenically prepared, surface-modified, structurally modified titanium dioxide and pyrogenically prepared, surface modified, structurally modified titanium dioxide mixed oxide according to claim 1, wherein the titanium dioxide prior to surface modification has a BET surface area of 10 to 150 m²/g.

8. The process according to claim 2, wherein the titanium dioxide is surface modified by spraying the titanium dioxide with water at a pH of 1 to 7, then spraying the titanium dioxide with a surface modification agent and then subjecting the titanium dioxide to a thermal treatment.

9. The process according to claim 2, wherein the titanium dioxide is surface modified by treating the titanium dioxide with a surface modification agent in vapour form and then subjecting the titanium dioxide to a thermal treatment.

10. The process according to claim 2, wherein the titanium dioxide after surface modification is structurally modified by mechanical means.

11. A method for preparing a sunscreen formulation comprising adding the titanium dioxide or mixed oxide of claim 1 to a sunscreen formulation.

12. The sunscreen formulation according to claim 4 containing one or more oil phases and one or more aqueous phases.

13. The sunscreen formulation according to claim 4 in the form of a microemulsion, stick, foam, solid emulsion, sprayable emulsion or hydroemulsion.

14. The sunscreen formulation according to claim 4 that is oil-free and in the form of an aqueous or alcohol solution.

15. A cosmetic or dermatological composition in the form of an emulsion containing the titanium oxide or mixed oxide according to claim 1.

16. The cosmetic or dermatological composition according to claim 15 which contains an emulsifier, the lipophilicity of which depends on temperature such that the lipophilicity increases when the temperature is raised and decreases when the temperature is lowered.

17. The cosmetic or dermatological composition according to claim 15 which further contains a gel-producer.

18. A cosmetic or dermatological product comprising a water insoluble substrate impregnated with the cosmetic composition according to claim 15.

19. A cosmetic tissue comprising a water insoluble substrate impregnated into the sunscreen formulation according to claim 4.

20. A composition comprising the titanium oxide or mixed oxide according to claim 1 and a synthetic or natural oil or wax.

21. The cosmetic or dermatological composition according to claim 15 in the form of a foamable composition.

22. The cosmetic or dermatological composition according to claim 21 which contains an alpha olefin-maleic enhydride copolymer.

23. The cosmetic or dermatological composition according to claim 22 which additionally comprises:
cyclic polysiloxanes of the type D 3, D 4, D 5, wherein
D 3, D 4 and D 5 are understood to represent cyclic polysiloxanes with 3, 4 or 5 units of the type —O—Si(CH₃)₂—
for example octamethylcyclotetrasiloxane=D 4

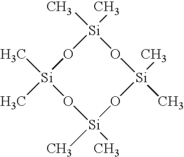

24. The sunscreen formulation according to claim 4 in the form of a Pickering emulsion.

25. The sunscreen formulation according to claim 24 which contains microfine particles of at least one of titanium dioxide, zinc oxide, iron oxide, boron nitride, starch derivative, talcum and latex.

26. The cometic or dermatological composition according to claim 15 which further contains a siloxane elastomer.
